(19) United States
(12) Patent Application Publication
(10) Pub. No.: US 2016/0159853 A1
(43) Pub. Date: Jun. 9, 2016

(54) APPARATUS FOR REFOLDING OF RECOMBINANT PROTEINS

(71) Applicant: BIOGENOMICS LIMITED, Maharashtra (IN)

(72) Inventors: Sanjay SONAR, West Thane, Maharashtra (IN); Archana KRISHNAN, West Thane, Maharashtra (IN); Nikhil GHADE, West Thane, Maharashtra (IN); Faiza SHAIKH, West Thane, Maharashtra (IN)

(21) Appl. No.: 14/905,438

(22) PCT Filed: Jul. 18, 2014
(86) PCT No.: PCT/IN2014/000476

§ 371 Date: Jan. 15, 2016

(30) Foreign Application Priority Data
Jul. 19, 2013 (IN) 2416/MUM/2013

(51) Int. Cl. C07K 1/113; B01J 19/24

(52) U.S. Cl. CPC C07K 1/1136; B01J 19/245; B01J 2219/24

(57) ABSTRACT

A machine or apparatus for refolding a protein of interest produced recombinantly in a host cell in form of inclusion bodies is provided. The apparatus includes an inclusion bodies (IB) solubilisation tank to solubilise the inclusion bodies; a plurality of refolding vessels or reactors arranged in series to receive a refolding feed; a first tank connected to the IB solubilisation tank to hold diafiltration buffer or agent; a first diafiltration cartridge having at least one permeate end and one retentate end, connected to the IB solubilisation tank, through a first retentate end to feed the refolding feed to the IB solubilisation tank; a second tank, connected to the first permeate end of the diafiltration cartridge to receive and recycle the refolding buffer and each of the refolding vessels, for supplying refolding buffer or agent to each of the refolding vessels.

### FIELD OF THE INVENTION

The present invention relates to production of recombinant proteins, and more particularly to a machine or apparatus for refolding of recombinant proteins from inclusion bodies produced in host cells.

### DESCRIPTION OF THE RELATED ART

Recombinant DNA (rDNA) technology has been used to clone, express and purify several proteins (e.g., mammalian proteins) of therapeutic or other economic value, such as human Insulin, human Insulin analogues, trypsin, Granulocyte Colony Stimulating Factor (G-CSF), Granulocyte Macrophage Colony Stimulating Factor (GM-CSF), etc. from prokaryotic as well as eukaryotic cells. However, the use of prokaryotic cells e.g. E. coli for manufacture of mammalian proteins is more widespread owing to the better scalability and cost-benefit economics of production of recombinant proteins.

One of the major challenges in expressing mammalian proteins in E. coli is that majority of expressed proteins is produced or expressed in form of inclusion bodies as unfolded inactive protein. The problem may be avoided by employing mammalian or insect cell lines; however, due to high expression levels of recombinant proteins in bacterial cells and reduced costs of production, bacterial cells are preferred.

The process of isolating an active and properly refolded recombinant protein from inclusion bodies includes, first of all, lysing bacterial cells to isolate inclusion bodies. Afterwards inclusion bodies are subjected to denaturing environment before exposing them to refolding buffer for obtaining isolated and refolded active recombinant proteins. The process of refolding may include treating the inclusion bodies with refolding buffer in infinite dilution based refolding process. The infinite dilution based refolding process is more preferred, since it provides a higher refolding efficiency, culminating into higher percentage of refolded active recombinant protein obtained, vis-a-vis unfolded recombinant protein in the inclusion bodies.

In context of infinite dilution based refolding processes, it is often desired to keep the concentration of the unfolded recombinant protein in the refolding buffer to be as low as possible. The infinite dilution is usually achieved by mixing a small quantity of inclusion bodies in substantially large quantities of refolding buffer. For example, if dilution concentration of 0.3 g/L or less is required dilution, there will be a need of 1000 litres of refolding buffer in a reactor tank to dilute 300 grams of inclusion bodies. This requirement of very high volume requires reactors to be large, well aerated with uniform or near ideal mixing. This leads to complication of purification processes, since for the subsequent steps (e.g., filtration, centrifugation, chromatography etc) needed to isolate refolded recombinant protein, will have very high batch volumes to start with or load onto various purification apparatus. Moreover, the process of moving the inclusion bodies and various other samples from one apparatus to another is cumbersome and time consuming activity. This further poses risk to aseptic process conditions, which are generally required for regulatory compliance.

Yet another method known in the art, for providing infinite dilution includes introducing small quantity of inclusion bodies in a moving stream of refolding buffer is disclosed in U.S. Pat. No. 4,999,422. The method even though largely reduces the quantity of refolding buffer that may be required for obtaining a refolded recombinant protein, the efficiency of doing so highly depends on the flow rates of the refolding buffer, which makes the process subjective and highly dependent on personnel operating the system.

In the current body of known art, there are many prior art references that disclose refolding apparatus or machine for obtaining refolded recombinant proteins from inclusion bodies obtained through bacterial fermentation.

EP1623988 discloses an apparatus for refolding proteins and a method of using an apparatus for refolding proteins that comprises various sections such as transport section, optical section, pH measurement section etc., within a single construct. The apparatus has limitations in scaling up for industrial processes. JP2003102494 discloses a method and apparatus for refolding protein that includes a denaturant dilution step in which a reversed micelle solution having a protein denatured with a denaturant is enclosed in an ultrafiltration membrane or dialysis membrane and soaked in a reversed micelle solution. The denaturant dilution in this apparatus requires large volumes of buffer due to inherent limitations of the apparatus.

WO200302590 discloses a tower-type protein folding reactor that includes a mixing chamber for holding a folding buffer; feeders for feeding denatured protein into the mixing chamber, and a mixing means capable of effecting high intensity mixing at each feed point in the mixing chamber. The protein folding reactor disclosed in this patent includes a tubular membrane. Further, the reactor extracts protein, denatures it and folds the protein integrally. The volume of buffer to dilute the denaturant, in order to affect refolding, is controlled by providing recirculation pipe and loops. However, despite vigorous mixing and control of buffer volume, some of the denaturant in the solution of denatured protein also passes from the tubular membrane into the mixing vessel, which is a limitation of this protein folding reactor. Further, the reactors or apparatus in the current body of art are not suitable for automation.

To add further to the limitations of the current body of art, the current refolding machines or apparatus lack integration of various processes, hence there is always a risk of contamination of the product (protein of interest), since due to lack of integration, the objective of maintaining aseptic conditions becomes subjective to the experience of personnel handling.

Furthermore, all the systems described above lead to localisation of unfolded proteins in the refolding feed and thereby restrict formation of stable intermediaries/intermediates. This further leads to low refolding efficiency, since the localization tends to tilt the refolding reaction towards aggregated conformation than refolding/refolded conformation.

Accordingly, there remains a need for a reactor or apparatus for enabling refolding of recombinant protein in an efficient way requiring low volume buffer for denaturation and refolding processes and integrating the operation in an efficient way with automated control.

### SUMMARY OF THE INVENTION

In view of the foregoing, the embodiments herein, provide an apparatus for refolding recombinant protein that enables capture of stable intermediates and is efficient in its usage of buffer solutions.

In an aspect, a machine or apparatus for refolding a protein of interest produced recombinantly in a host cell in form of inclusion bodies is provided. The machine includes an inclusion bodies (IB) solubilisation tank to solubilise the inclusion bodies isolated from the host cell; a plurality of refolding vessels or reactors arranged in series and connected to the IB solubilisation tank to receive a refolding feed; a first tank connected to the IB solubilisation tank; the tank holding diafiltration buffer or agent, which is pumped into the IB solubilisation tank; a diafiltration cartridge, having one permeate end and one retentate end; the diafiltration cartridge connected to the IB solubilisation tank, through a first retentate end, to feed the refolding feed to the IB solubilisation tank; a second tank connected to each of the refolding vessels for supplying refolding buffer or agent to each of the refolding vessels; the second tank connected to a first permeate end of the diafiltration cartridge to receive and recycle the refolding buffer.

The apparatus further includes a second diafiltration cartridge having at least one retentate and one permeate end, and connected to each of the refolding vessels or reactors via the retentate end, and to the second tank via said permeate end.

The apparatus further includes a third tank, connected to the refolding vessels for collecting concentrated and refolded protein. The IB solubilisation tank of the apparatus further includes probes for measuring pH, conductivity, level, temperature and rotor speed.

In the apparatus, the refolding feed is mixed with the refolding buffer and is first pumped in first refolding vessel at time, t=0 and in remaining vessels, from the series of refolding vessels, in a continuous manner at an equal interval that equals time required for diafiltration of refolded protein in first or second diafiltration cartridge.

### BRIEF DESCRIPTION OF THE DRAWINGS

For a more complete understanding of the embodiments herein, reference should now be made to the embodiments illustrated in greater detail in the accompanying drawings and described below by way of examples:

FIG. 1 illustrates a system view of an apparatus or assembly for refolding recombinant proteins, according to an embodiment herein;

FIG. 2 illustrates a system view of the apparatus or assembly for refolding recombinant proteins, with one filtration cartridge, according to an embodiment herein;

FIG. 3 illustrates RP-HPLC chromatogram of human Pro-Insulin, final hour refolding at concentration of 0.3 mg/ml. as refolded in the apparatus of the embodiment herein;

FIG. 4 illustrates RP-HPLC chromatogram of human Pro-Insulin refolded at 0.3 mg/ml and concentrated upto 1 mg/ml, as refolded in the apparatus of the embodiment herein; and

FIG. 5 illustrates the time difference in obtaining same amount of refolded protein, using same amount of refolding buffer, when different number of vessels are used for refolding, according to the embodiments described herein.

### DETAILED DESCRIPTION OF THE INVENTION

As required, detailed embodiments are disclosed herein; however, it is to be understood that the disclosed embodiments are merely exemplary, which can be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure. Further, the terms and phrases used herein are not intended to be limiting but rather to provide an understandable description of the invention.

The terms “a” or “an”, as used herein, are defined as one or more than one. The term “plurality”, as used herein, is defined as two or more than two. The term “another”, as used herein, is defined as at least a second or more. The terms “including” and/or “having”, as used herein, are defined as comprising (i.e., open language).

As mentioned, there is a need to develop a refolding apparatus that enables generation and capture of stable intermediates (henceforth, referred to herein as refolding feed, unless otherwise mentioned), which when immediately subjected to refolding process provides higher refolding efficiency. As is known, in terms of reaction kinetics, the two-state model of refolding of recombinant protein, may be better represented by the diagram below:

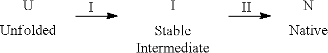

First stage includes formation of an intermediate I from the unfolded state U, the second stage that follows include formation of native state of the recombinant protein from the intermediate species I which is highly unstable and therefore tends to move reaction backwards towards unfolded form U. The apparatus described herein enables processes that enable formation of stable intermediates I₁ which have more tendency to move reaction forwards towards native conformation. The following model describes stable intermediate I₁:

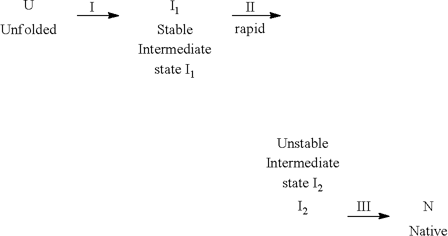

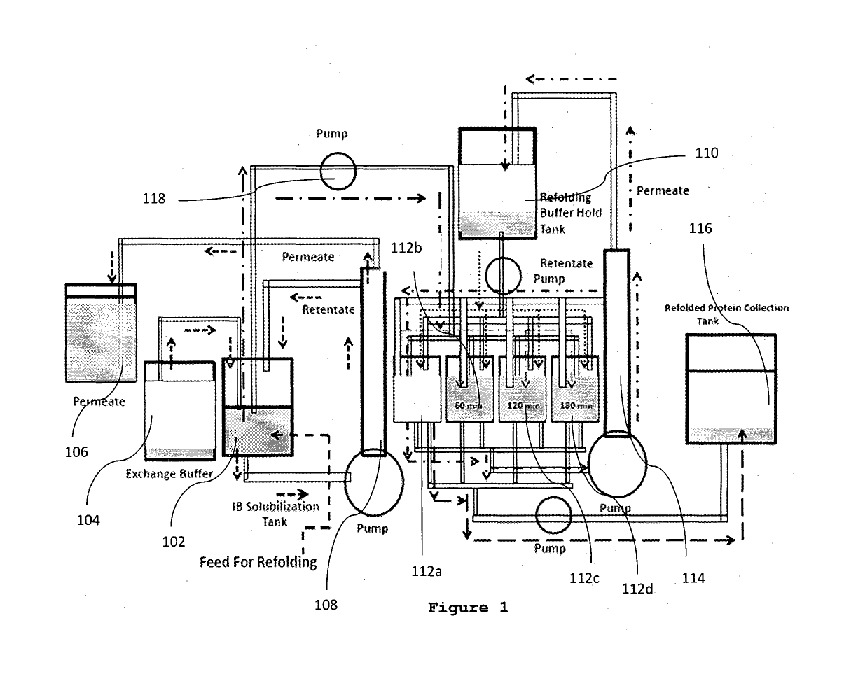

The apparatus as illustrated in FIG. 1 integrates the process of inclusion body solubilisation, trapping precursor (stable intermediates) for refolding and subsequently subjecting it to refolding for obtaining refolded recombinant protein. The apparatus of FIG. 1 includes a inclusion bodies (IB) solubilisation tank (102), a first buffer tank (104), a permeate holding tank (106), a first diafiltration cartridge (108), a second buffer tank (110), a number of refolding vessels or reactors (112a, 112b, 112c, 112d), a second diafiltration cartridge (114), a refolded protein collection tank (116), and a number of pumps (118).

The IB solubilisation tank (102) is used to hold and solubilise inclusion bodies or unfolded target protein obtained from the fermentation of host cells. The inclusion bodies include the protein of interest, among other proteins, in unfolded form or conformation. The IBs may be added manually to the IB solubilisation tank. Alternatively, the IBs may be added with the help of pumps designed for the purpose. Further, diafiltration agents or buffer are added to the IB solubilisation tank either manually or automatically through pumps taking feed from a tank (not shown in figure) containing the diafiltration agent or buffer.

The solubilising agent or buffer may include Dithiothrietol or any diafiltration or reducing agent. The IB solubilisation tank may be provided with at least one impeller for stirring the mixture of IBs and diafiltration agent or buffer. The IBs are held in the solubilisation tank (102) for duration of time required to achieve complete or near-complete solubilisation of inclusion bodies. Post complete reduction and solubilisation of IBS in the IB solubilisation tank (102); buffering agents may be added to bring the pH of the solubilised IBs to a desired level. In one embodiment, the pH of the solubilised IBs is brought at an acidic pH. In a preferred embodiment, the pH is brought at 3.

The IB solubilisation tank (102) further includes probes for measuring and controlling pH, conductivity, temperature, revolutions per minute (rpm) (of rotor). In one embodiment, the IB solubilisation tank (102) is made of a jacketed vessel having a level sensor with provision for multiple inputs. Post reduction or solubilisation of IBs in the IB solubilisation tank (102), the solubilised IBs may be filtered through the first diafiltration cartridge or cartridge (108) to obtain a feed for refolding the protein of interest in the IBs. For passing or pushing the solubilised IBs through the first diafiltration cartridge (108) the pump (118), which pumps solubilised IBs from the IB solubilisation tank (102) to the diafiltration cartridge or cartridge (108) as shown in FIG. 1, may be used. In one embodiment, the pump (118) is a peristaltic pump.

The first buffer tank (104) holds exchange buffer for the purpose of diafiltration. The exchange buffer may include 8M Urea, 20 mM glycine at pH 3.0. The diafiltration cartridge (108) of a particular molecular weight cut-off (as per the protein of interest in the IBs) may be selected and assembled. There may be multiple criteria for selecting membrane or cartridge for the purpose. For example, in an embodiment, the cartridge is selected based on a common rule that molecular weight cut-off should be 3 times lower than the molecular weight of the molecule/protein of interest. The weight cut-off depends on the protein of interest.

As soon as the process of diafiltration starts at the first diafiltration cartridge (108), flow rate at the permeate end (as shown FIG. 1) is detected (through appropriate probes) and adjusted to match with the feed flow rate of the exchange buffer. The permeate pipe (or end) and the retentate pipe (or end) (as shown in FIG. 1) is connected to the first diafiltration cartridge (108) at the cross-section, which is opposite to the cross-section where the feed and exchange buffer enters the first diafiltration cartridge (108).

The retentate and permeate end or pipe is additionally provided with probes or detectors such as pH, conductivity meter, UV sensor etc in order to monitor the retentate and permeate from the diafiltration cartridge (108).

According to the embodiments provided herein, the diafiltration is stopped after 5× buffer exchange is achieved. The buffer exchange volume may be reduced or increased as may be desired for different molecules or proteins. Alternatively, upon noticing consistent drop in UV readings (taken through the UV sensors) at retentate end which shows reducing concentration of the reducing buffer or agent; the diafiltration process may be aborted. The abortion of the diafiltration may be manual or it may be designed to be automated.

After the completion of the process at the first diafiltration cartridge (108), a precursor or feed for refolding containing stable intermediates that enable high refolding is obtained. Further, the refolding feed remaining in the tubing or on the cartridge is also drained and fed to the IB solubilisation tank (102).

The second buffer tank (110) holds refolding buffer or agent. The quantity or volume of the refolding buffer may be calculated based on the dilution that may be required to achieve refolding of the recombinant protein. The second buffer tank (110) then supplies the refolding reactor or vessels (112a-112d) with the refolding buffer in equal quantity through peristaltic pump(s) or other means for pumping. The refolding reactor or vessels (112a-112d) may have provision for pH and conductivity probes. Further, the refolding vessel (112) includes a jacket, which allows temperature control. In one embodiment, the jacket allows temperature control between 4° C. and 24° C. The refolding vessel (112) may further include an air sparger and a level sensor.

After the refolding vessels (112a-112d) have been filled with refolding buffer, the precursor for refolding or the refolding feed, as stored in the IB solubilisation tank (102), is supplied to the first refolding vessel or reactor (112a) at time zero i.e. at the beginning of the process of refolding. The other vessels (112c-112d) are supplied with refolding feed at equal intervals determined by the diafiltration time of the protein of interest at the second diafiltration cartridge (114). The second diafiltration cartridge (114) is connected, via its permeate end, to the second buffer tank (110). The second diafiltration cartridge is further connected to the refolding vessels (112a-112d) via its retentate end. The mixture of refolding buffer and the refolding feed is held in the refolding vessels (112a-112d) until refolding in each of them is achieved, on individual basis, before introducing or feeding the mixture to the second diafiltration cartridge (114).

Based on this arrangement, the mixture in the first vessel (112a) will be first subjected to diafiltration at the second diafiltration cartridge, while other vessels will continue to incubate the mixture to refold unfolded recombinant protein. Upon diafiltration, the permeate i.e. the refolding buffer is fed back to the second buffer tank (110), whereas the retentate i.e. the refolded protein is pumped back into the refolding vessels (112a) for concentrating the refolded protein. From there, the refolded and concentrated protein is collected separately in the protein collection tank (116) which is connected to all the refolding vessels (112a-112d). The process repeats until mixture in all the four vessels has been fed to the second diafiltration cartridge (114) for concentrating refolded protein in the retentate, from the second diafiltration cartridge (114), in the refolding vessels (112a-112d).

Simultaneously, while one refolding vessel, for example (112a), is emptied of its content i.e. the mixture of refolding buffer and refolding feed has been entirely subjected to the second diafiltration cartridge (114), the vessel (112a) is fed again with buffer from the second buffer tank (110), which now contains recycled or recovered buffer (permeate from the second diafiltration cartridge (114)) from the first diafiltration (of the mixture in the refolding vessel (112a)). The retentate from the second diafiltration cartridge (114) (for each cycle pertaining to each refolding vessel) is collected back in the refolding vessels (112a-112d) where the refolded protein (in the retentate) is concentrated. The refolding vessels (112a-112d) are connected to the refolded protein collection tank (116) where the concentrated protein is pumped from the refolding vessels (112a-112d) and collected accordingly.

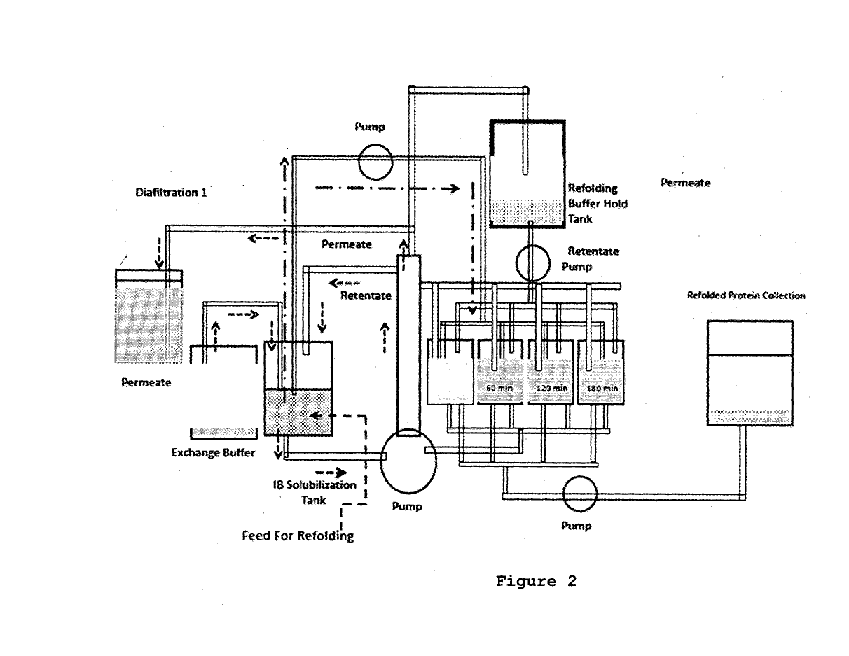

FIG. 2 illustrates a system view of the apparatus or assembly for refolding recombinant proteins, with one filtration cartridge, according to an embodiment herein. The apparatus in FIG. 2 is based on same principles of process design as the apparatus of FIG. 1. However, the apparatus of FIG. 2 is another embodiment that embodies the principles of process and system design for the apparatus of FIG. 1. The apparatus of FIG. 2 has one diafiltration cartridge to carry out exchange with the exchange buffer of the first buffer tank (104) to prepare precursor or feed for refolding, and to carry out diafiltration for purification and isolation of refolded recombinant protein, which may also be performed in the second diafiltration cartridge as explained above.

The apparatus of FIG. 1 and FIG. 2 have provisions for a number of pumps wherever necessary for pumping a fluid or buffer into one vessel from another. However, the flow rate of the pumps at the retentate end or pipe and feeding end or pipe of the diafiltration cartridges is matched and kept same, so as to enable better preparation of refolding feed as well as better purification and isolation of the refolded protein.

The apparatus for refolding of recombinant protein, as shown in FIG. 1 and FIG. 2, is made as a single closed unit embodying all the constituents of the apparatus as described above. Alternatively, the individual constituents of the refolding apparatus of the embodiments herein may be assembled in an open unit fashion, according to the principles of the process and system design of the embodiments disclosed herein.

One of the key advantages of the refolding machine or apparatus described herein is that it enables immediate refolding of the stable intermediates that are produced in the apparatus, since the process of preparing precursor for refolding or refolding feed (having stable intermediates) is integrated with process for refolding, thereby enabling automation and greater control of the process. Another advantage of the current refolding apparatus is that it employs minimal amount of buffer for refolding recombinant proteins by the dilution based methods while maintaining infinite dilution, which is required for optimal refolding of proteins.

The apparatus with various probes and detectors at the key junctions or connections of the vessels enable greater monitoring and control of the reaction process.

The machine or apparatus receives as input the inclusion bodies that comprise unfolded recombinant proteins among other proteins, and generates output of purified and isolated refolded recombinant protein, thereby signifying the amount of integration achieved by the embodiments of the refolding apparatus described herein. In another embodiment, the apparatus is connected to a controller or a processor that automates the various controls of the process described herein.

The working of the refolding apparatus as described above in FIG. 1 (and through an alternate embodiment in FIG. 2) is explained below by a way of example. However, the current example is non-limiting and the processes and its parameters may be scaled up as desired.

### EXAMPLES

### Example 1

### Refolding Human Pro-Insulin Using the Apparatus as Described Herein

2000 mg of inclusion bodies were transferred to and held in the IB solubilisation tank (102). 200 ml of 8 M Urea solution was added to the IB solubilisation tank (102) for solubilising IBs. The urea solution may be pumped from a tank (not shown in FIG. 1) to the IB solubilisation tank (102) for solubilising IBs in presence of 0.1 M DTT, which was added manually. The pH of the IBs in the IB solubilisation tank (102) was adjusted to 3.0.

After the IBs were solubilised, the solubilised IBs were subjected to diafiltration in the first diafiltration cartridge (108) against buffer consisting of 8 M Urea, 20 mM Glycine, pH 3.0. The buffer was pumped from the first buffer tank (104). The diafiltration was completed after 5× exchange and the precursor for refolding was generated. The precursor for refolding was highly stable intermediates with higher tendency to refold to produce refolded proteins. The permeate from diafiltration was stored in the permeated holding tank (106), whereas the retentate i.e. the precursor was sent back into IB solubilisation tank (102).

500 mg of precursor was pumped in to each of the refolding vessels or reactors (112a-112d). The refolding buffer was pumped into the refolding vessel (112a) from the second buffer tank (110) at 0 hour, such that the concentration of the precursor in the refolding vessel (112a) was 0.3 mg/ml. The total refolding volume was 1666 ml in the refolding vessel (112a). Based on the permeate flow rate of 13 ml/min, it was calculated that concentrating the protein back to 1 mg/ml would require 90 min. Hence, each of the subsequent refolding vessels (112c-112d) was fed with the precursor at equal interval of 90 minutes from the filling of the previous refolding vessel with precursor and the refolding buffer.

The refolding completion time for the molecule was determined to be 8 h. The concentration process was initiated for the first refolding reaction (of the vessel 112a) at the end of 8 h. 1166 ml of permeate was collected from each refolding reaction (of the vessels 112a-112d) in the second buffer holding tank (110). The pH of the recycled buffer was checked and again pumped to the refolding vessel (112a) and the refolding buffer was again pumped into the refolding vessel (112a) such that the concentration of precursor was 0.3 mg/ml. In the second cycle of refolding, 350 mg of protein was subjected to refolding and the above described process was repeated for other vessels (112c-112d) in the manner described earlier, until the refolded recombinant protein was obtained. No additional refolding buffer was added to the second buffer holding tank (110), since only recycled buffer was redistributed for subsequent refolding cycles.

By traditional method and systems, 6666 ml of refolding buffer volume would be consumed for refolding of 2000 mg of refolding feed (referred to herein also as precursor). By using the apparatus described herein, a total 5582 mg of protein was refolded using the same initial volume of refolding buffer i.e. 6666 ml by recycling the permeate buffer in the apparatus.

### Example 2

### Refolded Efficiency of Human Pro-Insulin in the Apparatus Described Herein

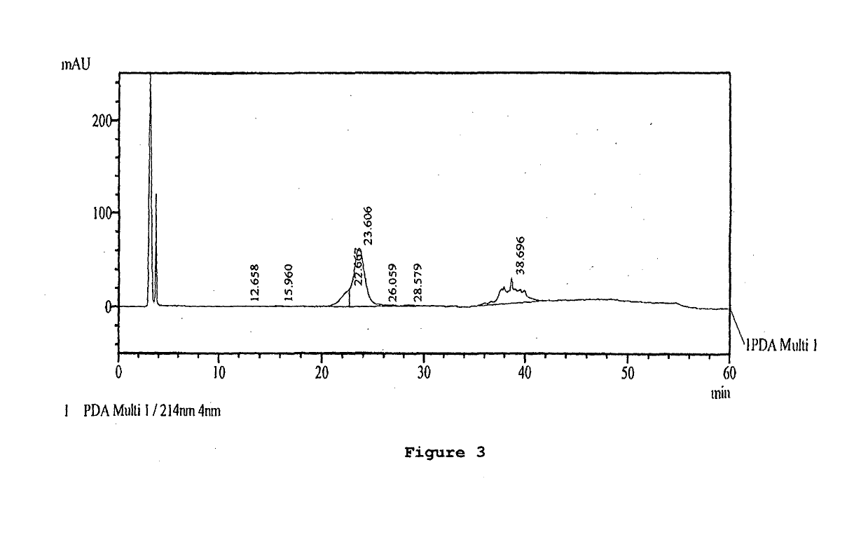

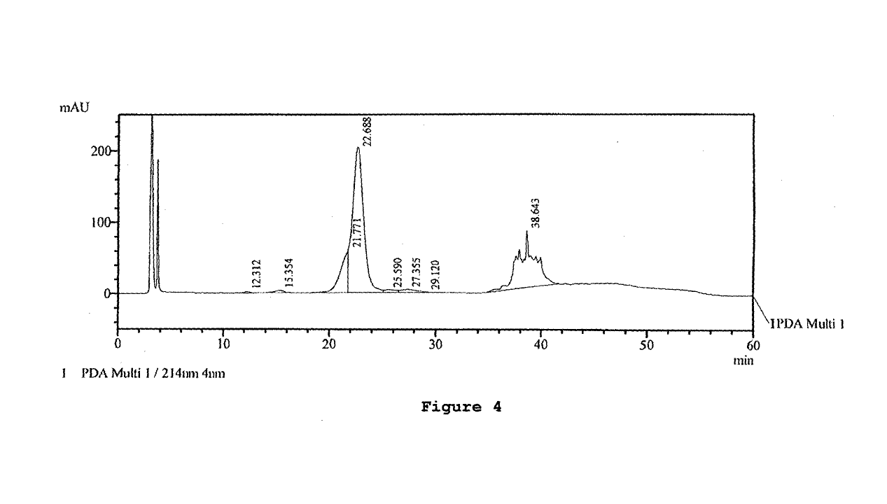

FIG. 3 illustrates RP-HPLC chromatogram of human Pro-Insulin, final hour refolding at concentration of 0.3 mg/ml. The refolding reaction was monitored and quenched after 8 hours on completion of folding, as a result of which 70% monomer of the refolding feed was obtained. FIG. 4 illustrates RP-HPLC chromatogram of human Pro-Insulin refolded at 0.3 mg/ml and concentrated upto 1 mg/ml. The yield of refolded Pro-Insulin post concentration of the monomer was 95% of the final hour refolded human Pro-Insulin.

### Example 3

### Apparatus with Single Refolding Vessel and Multiple Refolding Vessels

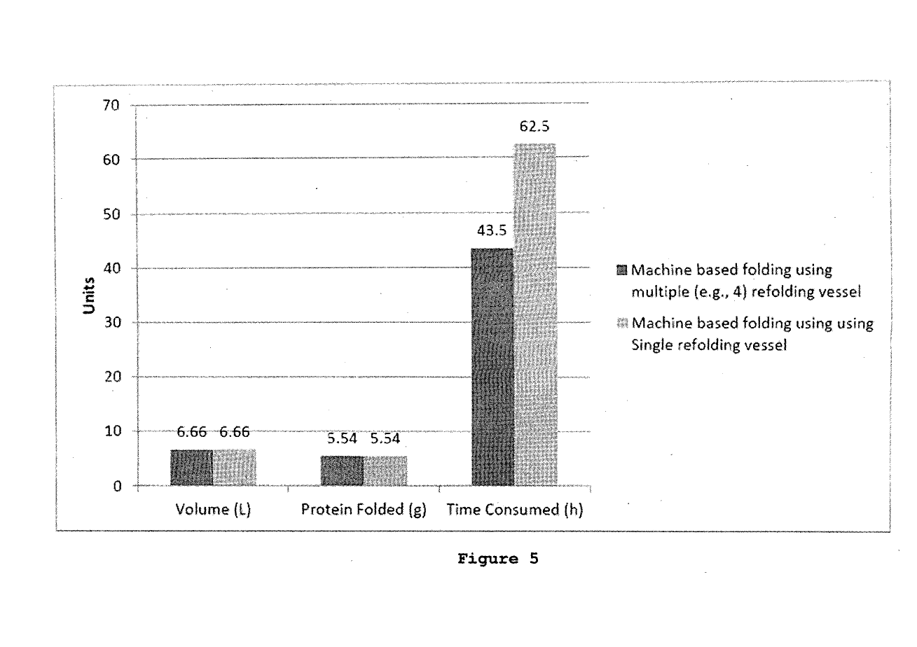

FIG. 5 illustrates the time difference in obtaining same amount of refolded protein, using same amount of refolding buffer, when different number of vessels are used for refolding according to the embodiments described herein. In one embodiment, a single refolding vessel was employed in order to refold recombinant protein. The time taken to refold 5.54 grams of protein was 62.5 hours. In another embodiment, and as shown in FIG. 1, four refolding vessels were employed that considerably reduced the refolding time to 43.5 hours.

1. A machine or apparatus for refolding a protein of interest produced recombinantly in a host cell in form of inclusion bodies, comprises:
a) an inclusion bodies (IB) solubilisation tank to solubilise said inclusion bodies isolated from said host cell;
b) a plurality of refolding vessels or reactors arranged in series and connected to said IB solubilisation tank to receive a refolding feed;
c) a first tank connected to said IB solubilisation tank; said first tank holding diafiltration buffer or agent, which is pumped into said IB solubilisation tank;
d) a first diafiltration cartridge, having at least one permeate end and one retentate end; said first diafiltration cartridge connected to said IB solubilisation tank, through a first retentate end, to feed said refolding feed to said IB solubilisation tank; and
e) a second tank connected to each of said refolding vessels for supplying refolding buffer or agent to each of said refolding vessels; said second tank connected to a first permeate end of said diafiltration cartridge to receive and recycle said refolding buffer.

2. The apparatus of claim 1 further comprises a second diafiltration cartridge having at least one retentate and one permeate end, and connected to each of said refolding vessels or reactors via a first retentate end of said second diafiltration cartridge and to said second tank via a first permeate end of said second diafiltration cartridge.

3. The apparatus of claim 1 further comprises a third tank connected to said refolding vessels for collecting refolded and concentrated protein.

4. The apparatus of claim 1, wherein said IB solubilisation tank further comprises probes for measuring pH, conductivity, level, temperature and rotor speed.

5. The apparatus of claim 1, wherein said IB solubilisation tank is made of a jacketed vessel.

6. The apparatus of claim 1, wherein said IBs are exchanged with said diafiltration buffer in said first diafiltration cartridge to obtain said refolding feed.

7. The apparatus of claim 6, wherein said refolding feed comprises a stable intermediate or precursor for refolding recombinant protein.

8. The apparatus of claim 1, wherein flow rate at said first permeate end of said first diafiltration cartridge is equal to feed flow rate of said diafiltration buffer from said first tank.

9. The apparatus of claim 2, wherein said permeate and said retentate ends of each of said first and second diafiltration cartridges are provided with probes for detecting pH, conductivity and UV absorption.

10. The apparatus of claim 2, wherein said refolding feed is mixed with said refolding buffer in first pumped in first refolding vessel at time, t=0.

11. The apparatus of claim 2, wherein said refolding feed is mixed with said refolding buffer in remaining vessels from said series of refolding vessels in a continuous manner with an equal interval that equals time required for diafiltration of refolded protein in first or second diafiltration cartridge.

12. The apparatus of claim 2, wherein diafiltration of refolded protein, held in said refolding vessels, is started either in first diafiltration cartridge or second diafiltration cartridge.

13. The apparatus of claim 12, wherein permeate having said refolding buffer from diafiltration of protein from said first or second diafiltration cartridge is pumped into said second tank.

14. The apparatus of claim 12, wherein retentate having refolded protein from said first or second diafiltration cartridge is pumped into said refolding vessels for concentrating said refolding protein.

15. The apparatus of claim 14, wherein said refolded and concentrated protein is pumped into said third tank for collection.

16. A method of refolding protein of interest produced recombinantly in a host cell in form of inclusion bodies, the method comprising refolding of said protein of interest in apparatus of claim 1.
